(19) United States
(12) Patent Application Publication
(10) Pub. No.: US 2018/0279940 A1
(43) Pub. Date: Oct. 4, 2018

(54) Disease Detection Device and Method for Detection of Abnormal Immunological Activity

(71) Applicant: James Campbell, Pfafftown, NC (US)

(72) Inventor: James Campbell, Pfafftown, NC (US)

(21) Appl. No.: 15/935,700

(22) Filed: Mar. 26, 2018

Related U.S. Application Data
(60) Provisional application No. 62/478,593, filed on Mar. 30, 2017.

(51) Int. Cl. A61B 5/00; A61B 5/01; G01J 5/00; G01V 8/12; G01V 3/08; G01V 1/00

(52) U.S. Cl. CPC A61B 5/41; A61B 5/01; A61B 5/70; G01V 1/001; G01J 5/0025; G01V 8/12; G01V 3/08; A61B 5/0059

(57) ABSTRACT

A disease detection device and method for detection or diagnosis of abnormal immunological activity is disclosed. The device is configured to detect the surface temperature of one or more hominid fingernails, thumbnails, or toenails by non-contact means and compare the temperature(s) detected to at least one second non-contact temperature measurement from the same subject, or to the nominal internal body temperature of the subject. The temperatures could be detected by non-contact electronic sensors, thermo-sensitive linear array sensors, or two-dimensional thermographic imagers. The device further comprises a computing device which executes instructions to compare the nail temperature(s) to the other temperature(s). The magnitude and polarity of the temperature difference of the nail(s) to the other temperature measurement(s) or the nominal internal body temperature of the subject indicates the presence of various pathological states, especially autoimmune, acute immune, drug-induced pathology, and various neurological, circulatory, traumatic, arthritic, and local infectious disorders of the hand.

### CROSS REFERENCE TO RELATED APPLICATIONS

This application claims the benefit of U.S. Provisional Application No. 62/478,593, filed Mar. 30, 2017, which is herein incorporated by reference in its entirety.

### TECHNICAL FIELD

The present invention generally relates to the field of diagnosis of abnormal immunological activity, and more particularly to a disease detection device and method for diagnosis of abnormal immunological activity.

### BACKGROUND

The detection and follow-up of human diseases of autoimmune type is often an expensive, uncomfortable, and complicated process. Various types of diagnosis method include symptom history; physical findings on gross clinical examination; chemical analysis of urine and blood chemistry; hematologic blood testing; tissue biopsy with specimen fixation, staining, and microscopic analysis; and careful microscopic examination of the small capillaries of the patient's nailbeds.

Although the foregoing methods have been proven to provide diagnostic information at least some of the time, none are suitable for quick and convenient screening. especially for patients who may be sensitive to contact and tactile sensations. The above stated diagnosis methods and their limitations are numerous. Symptom history during active autoimmune disease is extremely variable and is often erroneously considered just the normal ageing process by the patient. Thus, symptoms such as night sweats, joint aches, fatigue, digestive or urinary disorders, or fleeting rashes are often not thought of as associated with ongoing autoimmune disease. Likewise, findings on physical examination such as a mild rash over the cheeks or joint pain on motion testing may go unnoticed. Routine urine analysis, though quick, convenient and of low cost, often does not reveal immunological disease. Blood chemistry and hematology testing require skilled personnel working in a highly technical laboratory to process a sample of the patient's blood. Further, these tests often provide equivocal results, at great financial cost.

Tissue biopsy generally requires patient anesthesia, a surgically-skilled practitioner, proper specimen handling, and a fully-equipped professional pathology service to embed, slice, stain, and microscopically examine the sample. Lastly, professional microscopic analysis of the patient's nail beds requires a cooperative patient who can keep very still, a microscope preferably with photographic capabilities, and a skilled practitioner who has undergone specialized training for this procedure. Also confounding results of traditional screening are the effects the anxiety that many patients have when procedures are invasive or require the patient to perform uncomfortable tasks, such as giving a urine or blood sample or holding perfectly still as a medical health professional searches for an optimal point of insertion of a diagnostic tool. These traditional methods are ill-suited for the majority of patients who are in neurocognitive decline or anxious about even routine medical procedures.

Previous inventions seeking to use non-invasive methods for immunological disease detection involve cumbersome, expensive equipment and significant contact with the patient. Often, a skilled practitioner must administer the diagnostic method. A patient who is already anxious may present difficult behavior when someone other than a family-member caregiver handles her or his person.

Notwithstanding the advancements made in the prior art in the field of diagnosis of abnormal immunological activity, there remains a need for a low-cost, non-invasive and low contact device to detect immunological irregularities that general caregivers and patients could use easily.

### SUMMARY

This summary is provided to introduce in simplified form concepts that are further described in the following detailed descriptions. This summary is not intended to identify key features or essential features of the claimed subject matter, nor is it to be construed as limiting the scope of the claimed subject matter.

The present invention provides devices and methods for detection of immunological irregularities in a patient, and in particular provides convenient and rapid detection and follow-up of such conditions. The disease detection device is configured to detect-active autoimmune disease and related immune conditions such as substance-induced vasculitis and acute systemic infection. In addition, certain neurological, circulatory, traumatic, arthritic, and local infectious disorders of the hand or foot may also be detected by the devices and methods of this invention.

In clinical human thermography, a minority of patients have distinctly non-normal thermal patterns in their hands. In these patients, portions of their fingers are often much warmer than the wrist, with the fingernails and thumbnails being the warmest areas in many cases. Toenails are also seen to be unusually warm in a portion of these patients, but toenail warmth is less often encountered than fingernail and thumbnail warmth. Comparing the medical histories of the patients showing abnormal nail warmth to patients with normal hand thermal patterns reveals a strong correlation of the abnormal fingernail warmth with autoimmune and/or acute immune conditions. The physiologic basis for abnormal fingernail warmth in the case of active immune conditions is probably due to dilation of Glomus Bodies which are anatomically present in much higher concentration in the nail beds as compared to elsewhere in the skin. Glomus Bodies are very sensitive arterial-venous shunts which may be under control of the immune complexes in the circulating blood as well as by nerve fiber signals from the sympathetic system, thus bringing warm arterial blood to the nail region while other skin areas such as the wrist may remain cool due to normal vasoconstriction.

In at least one embodiment of the invention, the device is configured to detect the surface temperature of one or more digits such as hominid fingernails, thumbnails, or toenails and compare the temperature(s) detected to at least one second non-contact temperature measurement from the same subject, such as the skin temperature of the pad of the digit, or to the nominal internal body temperature of the subject. The temperatures may be detected by non-contact electronic sensors, thermo-sensitive linear array sensors, or two-dimensional thermographic imagers. The device further comprises a computing device comprising instructions to compare the nail temperature(s) to the other temperature(s). The magnitude and polarity of the temperature difference of the nail(s) to the other temperature measurement(s) or the nominal internal body temperature of the subject indicates the presence of various pathological states, especially autoimmune, acute immune, drug-induced pathology, and various neurological, circulatory, traumatic, arthritic, and local infectious disorders of the hand.

In at least one embodiment, the present invention provides a disease detection device and method utilizing temperature sensors. The device comprises a housing with an opening at a front end thereof to enable a user to insert a digit without contacting the housing. The housing comprises a top side, a bottom side opposite to the top side, a first side and a second side disposed opposite to the first side. A detection unit disposed within the housing is configured to detect insertion of a digit in the housing. At least two temperature sensors are disposed within the housing. These sensors comprise a first temperature sensor disposed at the top side of the housing for measuring a temperature of nail surface of a user, and a second temperature sensor disposed at the bottom side of the housing for measuring a temperature of a skin surface of the user. The housing is configured to shield the first and second temperature sensor from infrared light. A computing device in communication with the digit detection unit and at least two temperature sensors is configured to receive and analyze the nail surface temperature and skin surface temperature for magnitude and polarity to detect the presence of pathological condition of the user.

In at least one embodiment, the present invention provides a disease detection device and method utilizing a plurality of thermal imagers. The device comprises an enclosure with an opening at a front end thereof to enable a user to insert an extremity such as a hand or foot without contacting the enclosure and at least two thermal imagers are disposed within or mounted on the enclosure. During the test, the extremity is inserted into the enclosure with the user's nails are upward. Alternatively, the device enclosure and/or the at least two thermal imagers may be rotated while the hand or foot is held in one position. The plurality of two thermal imagers comprise a first thermal imager configured to detect nail surface temperatures, and a second thermal imager configured to detect pad temperatures. A computing device in communication with the plurality of least two thermal imagers is configured to analyze the nail surface temperatures and pad temperatures from at least two thermal imagers to detect the presence of pathological condition of the user. The device further comprises a detection unit in communication with the computing device to detect the presence of hand within the enclosure.

In at least one embodiment, the present invention provides a disease detection device and method utilizing a plurality of at least four thermal imagers. The device comprises an enclosure with an opening at a front end thereof to enable a user to insert a hand without contacting the enclosure and at least four thermal imagers are disposed within the enclosure. The plurality of least four thermal imagers comprises a first thermal imager configured to detect the nail surface temperatures, a second thermal imager configured to detect pad temperatures, and a third thermal imager and fourth thermal imager configured to detect the thumb surface or thumb pad temperatures. A computing device in communication with the at least four thermal imagers is configured to analyze the nail surface temperatures and pad temperatures from the at least four thermal imagers to detect the presence of pathological condition of the user. The device further comprises an extremity detection unit in communication with the computing device to detect the presence of an extremity within the enclosure.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 is a schematic drawing illustrating the disease detection device used on a user's digit.

FIG. 2A is a disease detection device including thermal sensor according to one embodiment of the present invention.

FIG. 2B is a cross-sectional view of disease detection device including thermal sensors according to one embodiment of the present invention.

FIG. 3A and FIG. 3B illustrates an electronic circuit of the disease detection device including thermal sensors according to one embodiment of the present invention. FIG. 3B is the continuation of FIG. 3A.

FIG. 4A is a front view of the tubular assembly having light beam interruption device according to one embodiment of the present invention.

FIG. 4B is a side view of the tubular assembly having detection unit PCB in one embodiment of the present invention.

FIG. 4C is a top view of the tubular assembly having PCB with temperature sensor according to one embodiment of the present invention.

FIG. 5 is an electronic circuit that detects the interruption of light beam according to one embodiment of the present invention.

FIGS. 6A and 6B are schematics each showing a disease detection device utilizing two thermographic imagers.

FIG. 6C is a schematic showing a disease detection device utilizing four thermographic imagers.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

As will be discussed in more detail infra, this application discloses various embodiments of a disease detection device and method for diagnosis of abnormal immunological activity. The present invention involves acquiring temperature data of nails and comparing the nail temperature with core body temperature or a skin surface temperature of the body to detect the presence of pathological condition. In many embodiments of the present invention, the device comprises single-thermopile or other chip devices, linear thermal arrays, or two-dimensional thermal arrays connected to a device that conveniently gathers the temperature data of fingernail and other temperature data. The device further comprises means to compute the temperature difference, and then displays the temperature differential in either analog or digital form automatically. Also, by examining the temperature data taken from multiple digits or nails, strong correlations to immune disease, drug reaction or other neurologic, circulatory, traumatic, or infectious processes may be computed and displayed without the need for a skilled human interpreter.

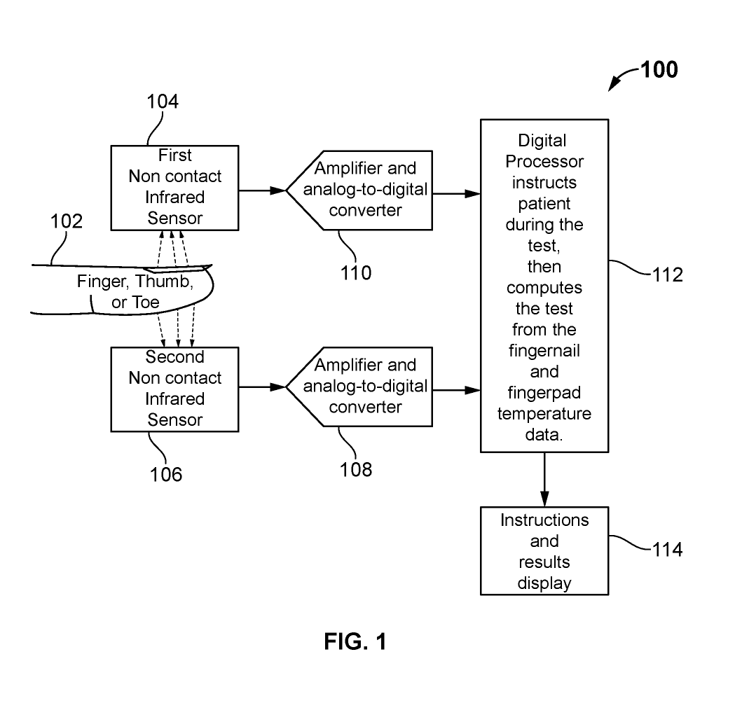

FIG. 1 is an environment 100 illustrating detection of immunological irregularities in patient according to one embodiment of the present invention. In an embodiment, the device comprising a placement area or measurement area to receive subject's digit 102 between a first non-contact infrared sensor 104 and a second non-contact infrared sensor 106. The first non-contact infrared sensor 104 is arranged such that it is present directly above the nail surface of the subject and the second non-contact infrared sensor 106 is arranged such that it is directly present below the skin surface of the subject, on receiving the subject's digit 102 in the placement area. The first non-contact infrared sensor 104 is configured to measure the temperature of the nail of the subject and the second non-contact infrared sensor 106 is configured to measure the temperature of the skin surface of the finger pad of the subject. An amplifier and analog-to-digital converter 108, 110 sends measured temperature data of the subject to the digital processor. The device further comprises a digital processor 112, which instructs and aids the patient during the procedure. The digital processor 112 computes the temperature data relating to the nail surface and finger pad—and displays instructions/result in a display unit 114.

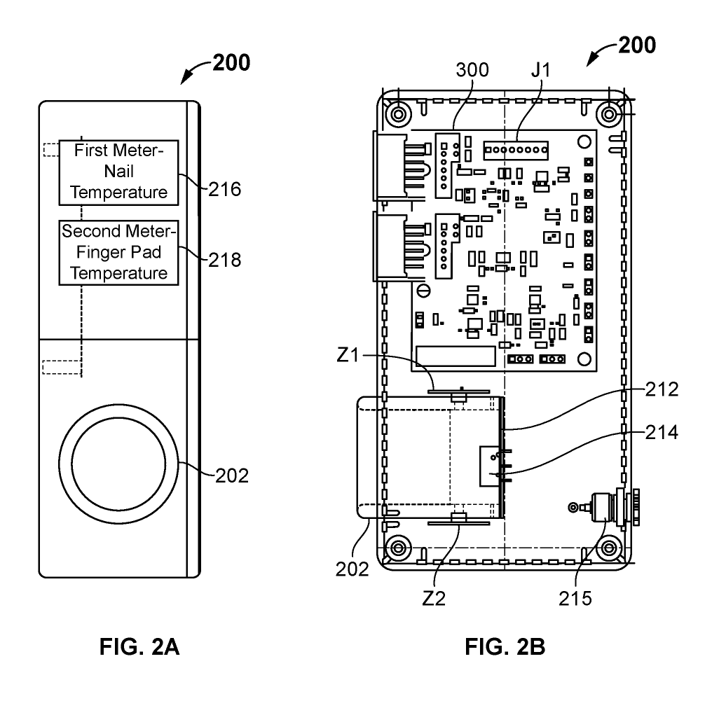

FIGS. 2A and 2B is a disease detection device 200 including thermal sensors according to one embodiment of the present invention. The disease detection device 200 comprises a housing 202 having an open end and at least two thermal sensors or temperature sensors disposed within or mounted on the housing 202. The housing 202 comprises a top 204, bottom 206, a collection of at least three side portions and a measurement area. The top portion 204 disposed opposite to the bottom portion 206. The collection of at least three side portions includes a first side 208, a second side 210 disposed opposite to the first side 208, a third side 212 disposed opposite to the open end of the housing 202. The open end is configured to receive the digit of the subject and place at the measurement area. In an embodiment, the housing 202 is a tubular assembly 202. The at least two thermal temperature comprises a first temperature sensor Z1 and a second temperature sensor Z2. The first temperature sensor Z1 is configured to measure a temperature of a nail surface of the subject, patient or user. The second temperature sensor Z2 is configured to measure a temperature of the skin surface of a finger or toe pad of the subject. The tubular assembly 202 is configured to shield the at least two sensors from external infrared light sources. The device 200 further comprises an electronic printed circuit assembly 300, 12V power connector 215, and two temperature display meters 216, 218.

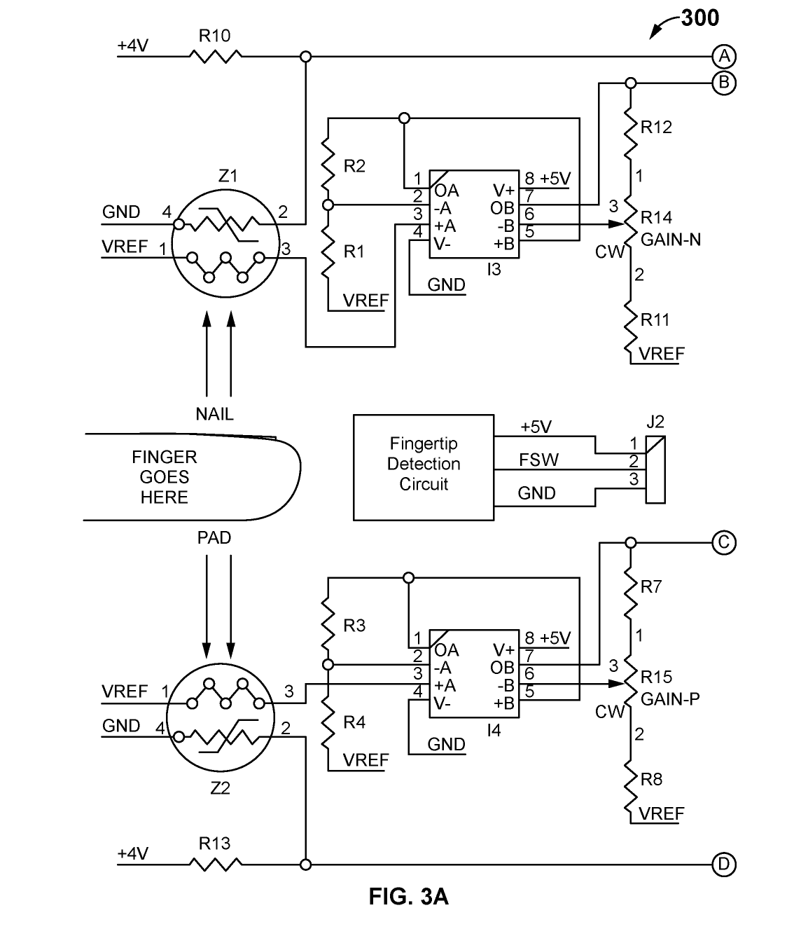

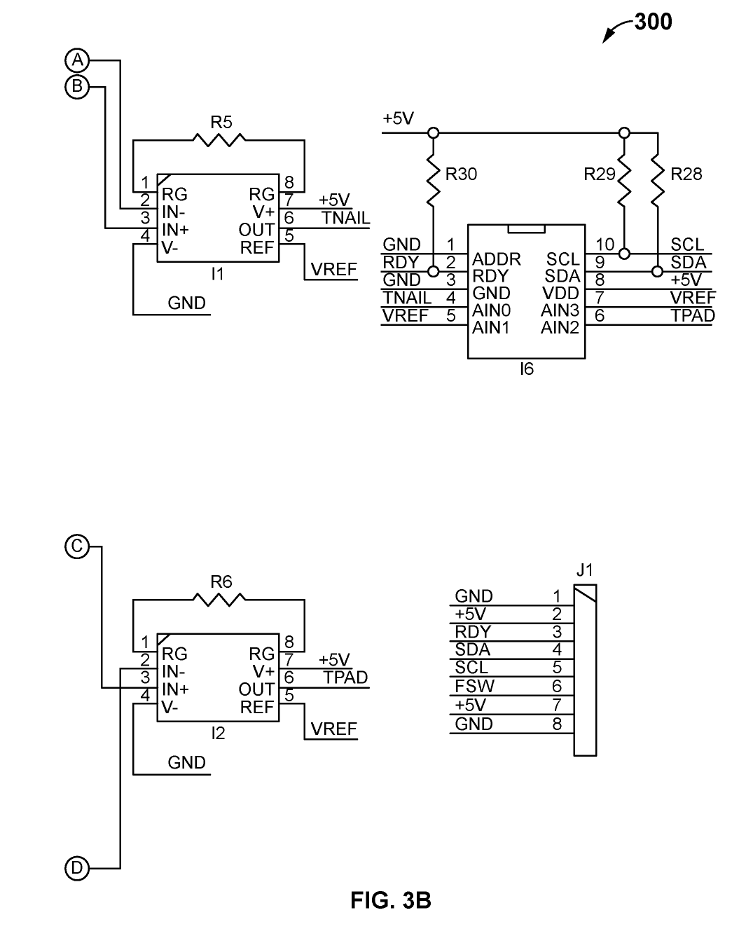

In the same embodiment, as shown in FIG. 2B, the first temperature sensor Z1 is disposed at the top portion 204 of the housing 202 and the second temperature sensor Z2 is disposed at the bottom portion 206 of the housing 202. A detection unit is disposed at the third side 212 of the housing 202. In at least one embodiment, the detection unit is a digit detection mechanical switch 214 to detect the presence of digit. The present invention further comprises a cable assembly to carry the electrical signal from the first and second temperature sensors Z1 and Z2 and the digit detection unit to an electronic circuit board 300 of the device 200, where the signals are detected, amplified and digitized, shown in FIG. 3. The data is then exported via a board connector J1 to a microprocessor for analysis and display.

FIG. 3A and FIG. 3B illustrates an electronic circuit 300 of the disease detection device 200 including thermal sensors according to at least one embodiment of the present invention. FIG. 3B is the continuation of FIG. 3A. The electronic circuit 300 acquires, amplifies, calibrates and digitizes the digit temperatures of the device 200. The thermal sensor device Z1 is comprised of an integrated thermopile, sensitive to infrared radiation in the 8 to 10-micron wavelength band of mammalian thermal emissions and a calibrated temperature-sensitive thermistor which monitors the case temperature of Z1. The infrared-sensitive thermopile of Z1 is aimed at the nail of the subject to detect the nail's surface temperature. The thermistor inside the case of Z1 forms a voltage divider with resistor R10. As the resistance of the internal thermistor of Z1 decreases with increasing case temperature, the voltage output from the voltage divider consisting of Z1 and R10 is negatively correlated with Z1's case temperature and is wired into the inverting input of instrumentation amplifier I1. The infrared-sensitive thermopile in Z1 produces a voltage proportional to the temperature difference produced by infrared radiation heating the front surface of the thermopile chip as compared to the temperature of the back surface of the chip which is monitored by the thermistor in Z1. The voltage generated by the thermopile is much smaller than the voltage produced by the thermistor inside Z1 and must be amplified by a factor of around 1000 by the precision noninverting operational amplifiers I3A & B so that the voltage output amplitude matches that of the output of the Z1-R10 voltage divider. As the thermal sensitivity of the thermopile in Z1 is dependent on factors such as the distance and orientation between the surface being measured and the case of Z1 and might vary from unit-to-unit, the amplification factor of I3A&B must be adjustable. This is accomplished by the variable resistor R14 (GAIN-N). The amplified voltage signal from I3B is wired to the noninverting input of instrumentation amplifier I1. Instrumentation amplifier I1 subtracts the negatively correlated thermistor voltage from the positively correlated thermopile output from I3B to produce a voltage output labeled TNAIL (Temperature of Nail) which is positively and linearly correlated with the actual surface temperature of the fingernail inserted into the device, independent of the case temperature of Z1. Voltage TNAIL may be scaled so that first meter displays 216 (shown in FIG. 2A) the actual nail temperature in Fahrenheit or Centigrade degrees. Voltage TNAIL is also digitized in Analog-to-Digital Converter 16. The digital data stream from 16 is transmitted by an Inter-Integrated Circuit Bus (“I2C Bus”) to the microprocessor via Board Connector J1.

The sensor Z2 is aimed to detect the surface temperature of the volar finger pad of the same finger as Z1. The thermistor inside the case of Z2 forms a voltage divider with resistor R13. As the resistance of the internal thermistor of Z2 decreases with increasing case temperature, the voltage output from the voltage divider consisting of Z2 and R13 is negatively correlated with the case temperature of Z2 and is wired into the inverting input of instrumentation amplifier I2. The infrared-sensitive thermopile in Z2 produces a voltage proportional to the temperature difference produced by infrared radiation heating the front surface of the thermopile chip as compared to the temperature of the back surface of the chip which is monitored by the thermistor in Z2. The voltage generated by the thermopile is much smaller than the voltage produced by the thermistor inside Z2 and must be amplified by a factor of around 1000 by the precision noninverting operational amplifiers I4A&B so that the voltage output amplitude from I4B matches that of the output of the Z2-R13 voltage divider. As the thermal sensitivity of the thermopile in Z2 is dependent on factors such as the distance and orientation between the surface being measured and the case of Z2 and may vary from unit-to-unit, the amplification factor of I4A&B must be adjustable. This is accomplished by the variable resistor R15 (GAIN-P). The amplified voltage signal from I4B is wired to the non-inverting input of instrumentation amplifier I2. Instrumentation amplifier I2 subtracts the negatively correlated thermistor voltage from the positively correlated thermopile output from I4B to produce a voltage output labelled TPAD (temperature of digit pad) which is positively and linearly correlated with the actual surface temperature of the volar pad inserted into the device, independent of the case temperature of Z2. Voltage TPAD may be scaled so that a second meter 218 (shown in FIG. 2A) displays the actual nail temperature in Fahrenheit or Centigrade degrees. Voltage TPAD is also digitized in Analog-to-Digital Converter 16. The digital data stream from 16 is transmitted by an Inter-Integrated Circuit Bus (“I2C Bus”) to the microprocessor via Board Connector J1.

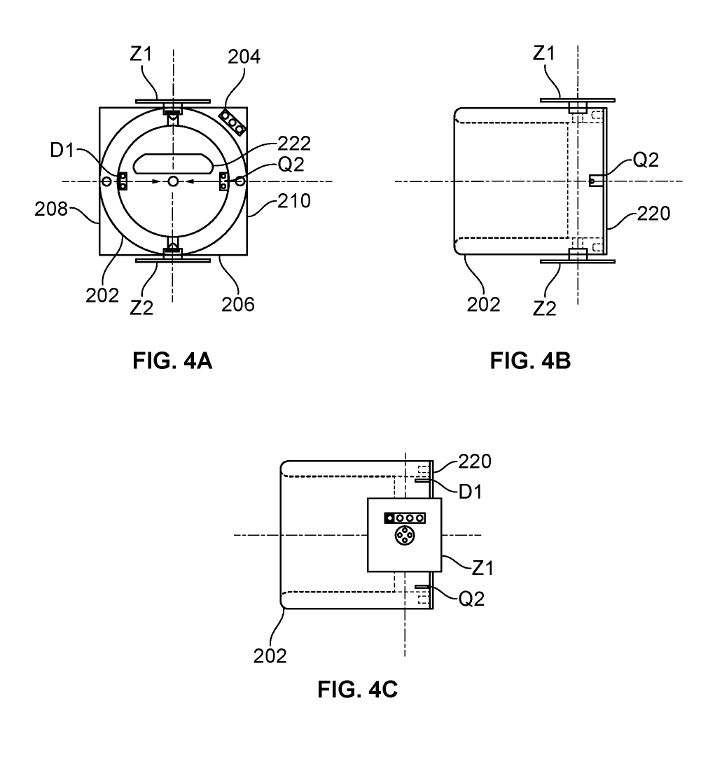

The digit detection unit sends signal FSW to the microprocessor, indicating the fingertip has been inserted between the two infrared sensors Z1 and Z2. Then the two temperature values TNAIL and TPAD are digitized by the Analog-to-Digital Converter 16 and stored in the microprocessor memory. Then TNAIL and TPAD are analyzed for magnitude and polarity and the results are used to detect the possible presence of pathological conditions. Signal FSW may also be used to display TNAIL and TPAD on first 216 and second meters 218, and then hold these values on the meters for reading. In an embodiment, the fingertip detection unit is at least any one of a mechanical pushbutton, capacitive sensing unit, ultrasonic digit detection unit or light beam interruption device. Referring to FIG. 4A-4C, detection of digit by light beam interruption unit comprises at least one light emitter diode or photo emitter diode D1 disposed at the first side 208 of housing 202 and configured to emit light; and at least one light-sensitive phototransistor Q2 disposed at the second side 210 of the housing 202 is configured to detect interruption of light emitted from the at least one diode D1 to detect the presence of finger. In an embodiment, the photo emitter diode D1 and the light-sensitive phototransistor Q2 are held on either side of the desired fingertip position by the NIR (Near-infrared) Finger Detector Printed Circuit Board (PCB) 220 as shown in FIG. 4C. Housing 202 is comprised of a black ABS (acrylonitrile-butadiene-styrene) tube with bore ridged, roughened, or threaded to reduce IR reflections.

The optical axis between D1 and Q2 is at right angles to the axis between the thermal detectors Z1 and Z2 to reduce any interference with thermal measurement. Such interference is further reduced by the filter windows built in to Z1 and Z2, which block visible or near-infrared (NIR) light from reaching the thermopile chips in these devices. The optical axis between D1 and Q2 is located so that the bed of the nail is directly below Z1 when the light beam between D1 and Q2 is interrupted. The NIR Finger Detector PCB 220 contains a slot 222 to accommodate a long nail, allowing the fleshy tip of the digit to touch the board. This assures that the nail bed is directly under the thermal sensor Z1 and the digit pad is directly above the thermal sensor Z2 at the time of the temperature measurement.

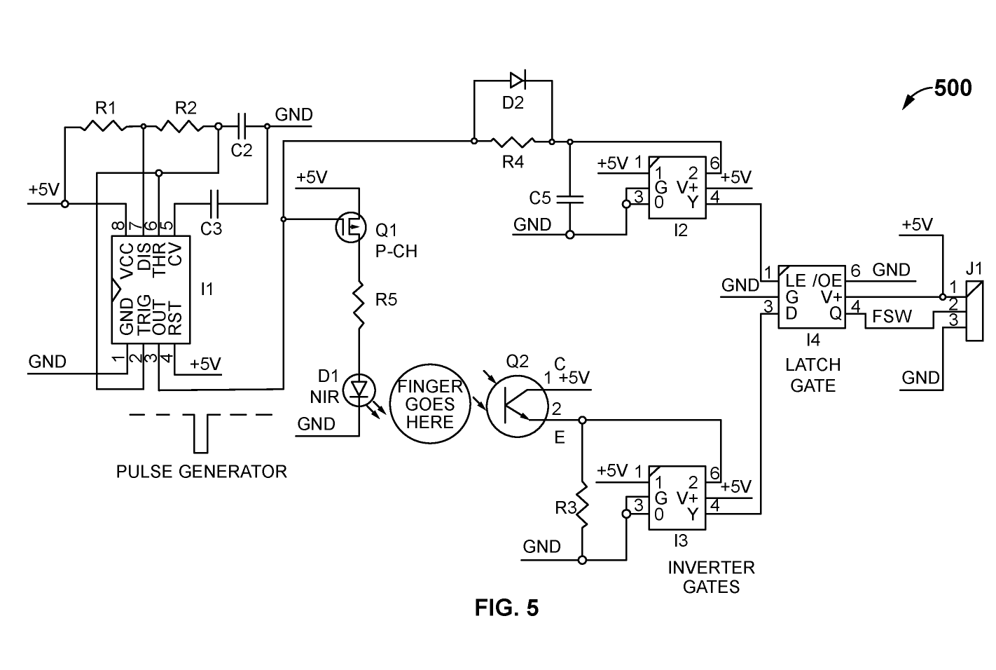

FIG. 5 is an electronic circuit 500 that detects the interruption of light beam according to one embodiment of the present invention. In an embodiment, the light beam interruption unit detects the interruption of small beam of light and converts the loss of light transmission from D1 to Q2 into the binary output signal FSW. The output signal FSW stays high when the light beam is interrupted and stays low with return of the light beam transmission. To save power compared to keeping D1 ON continuously, D1 is pulsed ON briefly at regular intervals, and the output of Q2 is latched high or low between the pulses.

A LM555 Timer integrated circuit I1 is connected to produce a negative-going 1-millisecond pulse every 150 milliseconds. Each short negative pulse turns on the P-channel MOSFET transistor Q1, energizing the near-infrared (NIR) emitting diode D1. D1 is oriented to send the short pulses of light toward the NIR-sensitive phototransistor Q2. The pulse of light turns Q2 ON, driving the input of inverter I3 high. The output of inverter I3 thus goes low. A circuit consisting of D2, R4, and C5 delays the input of inverter I2 going low until the output of inverter I3 settles high or low. When the output of inverter I2 goes high, it latches the output of inverter I3 into the FSW output of the integrated circuit latch I4. When the output of inverter I2 goes low at the end of the 1-millisecond pulse from I1, the FSW signal is latched by I4 and cannot change until output of inverter I2 goes HIGH again. Thus, when an opaque object such as a fingertip blocks the light transmission from D1 to Q2, the output signal FSW goes high for as long as the light beam is blocked, then FSW returns and stays low when the opaque object is removed from the light path.

In at least one embodiment of the invention, the device is configured to be highly light-weight and portable so that a frail patient or caregiver could easily maneuver and operate it. In such embodiments, the materials comprising the housing of the device are non-conductive plastic. In at least one embodiment, Acrylonitrile-Butadiene-Styrene is used for the case for device 200 and sensor housing 202 and a fiberglass printed circuit 300 for light weight and electrical insulation such that the entire weight of the device weighs no more than 1 lb. (480 gm).

In at least one embodiment, the present invention provides a method for detecting the presence of pathological conditions. The disease detection accuracy and specificity of the invention as described can be further enhanced by repeating the described temperature difference detection on all the subject's digits, then via either manual or computerized means, computing an average value and polarity of the nail-to-pad and nail-to-core body temperatures, a maximum value and polarity of the nail-to-pad and nail-to-core body temperatures, the number of nail-to-pad and nail-to-core body temperatures greater or less than a certain value, detecting which digits show normal versus abnormal nail and/or pad temperatures, and by other algorithms. By determining which digits show normal or abnormal temperature measurements, various neurological, circulatory, traumatic, or infectious processes may also be detected by the methods of this invention.

A method comprising a step of providing a digital computing device 112 in communication with the disease detection device 200 via Board Connector J1 is herein described. This digital computing device executes programmable instructions to execute the following steps: The computing device is configured to interact with users to provide instruction to aid during the test. At another step, the computing device instructs the subject via visual or audible means 114 to enter a vasoconstricted state by resting while lightly clothed in an area of ambient temperature at least 20° F. (11.1° ° C.) below the nominal body temperature of the user for about 15 minutes as timed by the computer program. During this time the program reminds the subject that the digits should be unclasped in free air and not touching anything. This temperature equilibration period could be shortened or waived by invoking a command to the computing device in case of repeat testing or an already vasoconstricted subject.

At another step, the computing device 112 instructs the subject by visual or audible means 114 to insert one digit at a time into the device measurement area. For example, the computing device might display visually or by voice command “Insert the left ring finger.” The digit sensing circuit 500, shown in FIG. 5, then sends a signal FSW through the board connector J1 indicating when the requested fingertip has been inserted and is ready for temperature measurement. The computing device then adds two or more data points to its memory, which includes the fingernail temperature TNAIL and the finger pad temperature TPAD of the digit that is being measured. At another step, if the finger of a subject cannot be tested perhaps due to amputation, congenital absence, severe deformity, or other problem, a means to signal the computing device, such as a button push or voice command, could be invoked by the subject.

The computing device then registers that the requested digit cannot be tested and instructs the subject to insert the next digit into the device 200. At another step, after measuring as many of a subject's digits temperatures as possible, the computing device examines the collected data and with the use of experimentally-defined parameters, look-up tables, artificial-intelligence algorithms, and/or other computational techniques, computes and displays the various diagnostic possibilities that could account for the detected thermal pattern. These results can then be printed, saved in memory, and/or transmitted via electronic means as appropriate for the medical case.

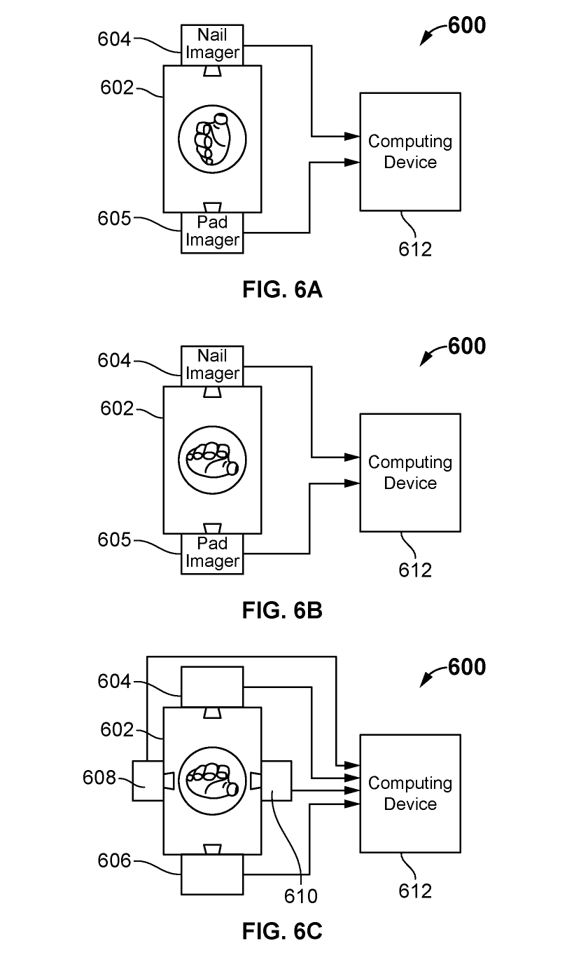

FIG. 6A and FIG. 6B show a disease detection device 600 utilizing thermographic imagers or other non-contact thermography devices according to one embodiment of the present invention. The thermography device 600 comprises an enclosure 602 with an opening at a front end thereof to enable the user or subject to insert a hand or foot without contacting the enclosure 602 and a plurality of thermal detection devices disposed within or mounted on the enclosure 602. The enclosure 602 is kept at or near the ambient temperature of the room. The enclosure 602 has a non-reflective inner surface with a thermal emissivity close to 0.99. The hand or foot of the subject is inserted into the enclosure 602 through the opening. The enclosure comprises a top side, a bottom side opposite to the top side, a first side and a second side disposed opposite to the first side. The embodiment shown in FIG. 6A and FIG. 6B, comprises two thermal detection devices. The hand of the subject is inserted into enclosure 602 with the thumbnail oriented so that it is visualized by thermal imager 604 and the thumb pad visualized by thermal imager 606 as shown in FIG. 6A. Then the hand is rotated 90 degrees as shown in FIG. 6B so that the fingernails are visualized by thermal imager 604 and the finger pads are visualized by thermal imager 606.

In another embodiment shown in FIG. 6C, four thermal imagers are disposed within or mounted on the enclosure 602. The upper imager or the first imager 604 disposed at the top side detects the nail temperatures; the bottom imager or the second imager 606 disposed at the bottom side detects the pad temperatures; and the two imagers on the sides or the third 608 and the fourth thermal imagers 610 disposed at the first and second sides, respectively, record the thumb nail or pad temperatures, depending whether the left or right hand is inserted into the enclosure 602. The ambient temperature is read by imaging the inner surface of the enclosure 602. As the temperature calibration of thermal imagers (604, 606, 608, 610) may be very prone to drift, the four imagers (604, 606, 608, 610) in this embodiment could be re-calibrated to a single temperature standard by imaging the inside of the isothermal enclosure 602 and correcting for any detected temperature differences among the imagers (604, 606, 608, 610) during the computational analysis. The use of four thermal imagers as shown in FIG. 6C requires more complicated hardware and computational analysis that the embodiment shown in FIG. 6A and FIG. 6B, the embodiment shown in FIG. 6C may be used with the hand inserted into enclosure 602 in any orientation, and does not require active rotation of the subject's hand as shown in shown in FIG. 6A and FIG. 6B.

In the same embodiment of the thermographic device 600 as shown in FIG. 6C, further comprises a computing device 612 in communication with a plurality of thermal imagers (604, 606, 608, 610), which is configured to analyze nail surface temperature and finger pad temperature from the plurality of thermal imagers to detect the presence of pathological condition of the user. The infrared thermal imagers (604, 606, 608, 610) sensitive to the 8 to 10-micron wavelength band take radiometric thermographic still images or video images of both sides of the subject's fingers and thumbs. Insertion of a hand or foot into the device 600 is detected by photoelectric beam interruption or reflection, ultrasonic means, or other non-contact means. The computing device 612 using either operator-guided or specialized image processing techniques selects the location of the finger and thumb nails and pads within the images and measures the temperature detected at each site. The subject's core body temperature is assumed from statistical data. The computing device 612 then takes the collected data and with the use of experimentally-defined parameters, look-up tables, artificial-intelligence algorithms, and/or other computational techniques computes and displays the various possibilities that could account for the detected thermal pattern. These results can then be printed, saved in memory, and/or transmitted via electronic means as appropriate for the case at hand.

In at least one embodiment, the present invention provides a method for detecting the thermal pattern of the subject utilizing the non-contact thermographic device 600 is disclosed. The attached computing device 612 with a specialized software program first instructs the subject to enter a vasoconstricted state by resting in an area of 68 to 70° F. (20 to 21° C.) ambient temperature while lightly clothed for about 15 minutes as timed by the computer program. During this time the computing device 612 reminds the subject that the hands should be unclasped in free air and not touching anything. This temperature equilibration period could also be shortened or waived by invoking a command to the computing device 612 in the case of repeat testing or an already vasoconstricted subject. Once the cool-down period is over or has been waived, the computing device 612 instructs the subject to insert one hand into the enclosure, where it is radiometrically imaged by the plurality of thermal imagers. Then the computing device 612 instructs the subject to insert the other hand, which is likewise thermally imaged. Once the thermal image data is sent to the computing device, it is then processed for image features and analyzed for thermal patterns of the nails, fingers, and hand compared to the nominal body core temperature. Differential possibilities for the detected thermal patterns are determined by the computing device 612 and the results are displayed, printed, or output to other devices for medical consideration.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

The invention claimed is:

1. A disease detection device, comprising:
a housing with an opening at a front end thereof to enable a user to insert a digit without contacting the housing, wherein the housing comprises a top side, a bottom side opposite to the top side, a first side and a second side disposed opposite to the first side;
a digit detection unit disposed within the housing is configured to detect presence of a digit;
at least two temperature sensors disposed within the housing comprising,
a first temperature sensor disposed at the top side of the housing for measuring a temperature of a nail surface of a user,
a second temperature sensor disposed at the bottom side of the housing for measuring a temperature of a skin surface of a pad of the user;
wherein the housing is configured to shield the first and second temperature sensor from infrared light;
a computing device in communication with the detection unit and at least two temperature sensors is configured to receive and analyze the nail surface temperature and skin surface temperature for magnitude and polarity to detect the presence of pathological condition of the user.

2. The disease detection device of claim 1, wherein the housing of the device is composed of lightweight non-conductive plastic and a plurality of electronic circuits in the device is made from fiberglass material so that the disease detection device weighs no more than one pound.

3. The disease detection device of claim 1, further comprising an infrared sensor assembly.

4. The disease detection device of claim 1, wherein the housing further comprises a slot for receiving the nail of the user.

5. The disease detection device of claim 1, wherein the digit detection unit is a mechanical switch.

6. The disease detection device of claim 1, wherein the digit detection unit is an ultrasonic digit detection unit.

7. The disease detection device of claim 1, wherein the digit detection unit is a capacitive sensing unit.

8. The disease detection device of claim 1, wherein the digit detection unit is a light beam interruption device.

9. The disease detection device of claim 8, wherein the light beam interruption device comprises
at least one light emitter diode disposed at the first side of the housing is configured to emit light; and
at least one light-sensitive phototransistor disposed at the second side of the housing is configured to detect interruption of light emitted from the at least one diode to detect the presence of finger.

10. A method to detect a presence of pathological condition of user, comprising steps of:
providing a disease detection device, comprising
a measurement area for receiving a digit of a user,
a digit detection unit for sensing the presence of a digit in the measurement area,
a first temperature sensor for measuring a temperature of a nail surface of the user,
a second temperature sensor for measuring a temperature of a skin surface of a pad of the user, and
a computing device in communication with the first and the second temperature sensors and the digit detection unit, configured to provide instruction to user to aid during a test procedure;
providing instruction to the user to undergo a vasoconstricted state;
inserting one digit at a time into the measurement area;
sensing the presence of a digit by the detection unit;
receiving a signal indicating the presence of the digit in the measurement area for measuring temperature data of the finger;
measuring a temperature of the nail surface and the skin surface of the user; and
analyzing the temperature of the nail surface and the skin surface by the computing device for magnitude and polarity to detect the presence of pathological condition of the user.

11. The method of claim 10, wherein the user undergoes the vasoconstricted state by resting in an area of ambient temperature at least 20° F. (11.1° ° C.) below the nominal body temperature of the user.

12. The method of claim 10, wherein the step of measuring temperature of nail surface and skin surface of the user is performed in all fingers of the user.

13. The method of claim 10, wherein if the finger of the user is amputated, the computing device is configured to instruct the user to place a different finger.

14. A disease detection device, comprising:
an enclosure with an opening at a front end thereof to enable a user to insert a hand or foot without contacting the enclosure,
wherein the enclosure comprises a top side, a bottom side opposite to the top side,
a first side and a second side disposed opposite to the first side;
an extremity detection unit disposed within the enclosure is configured to detect presence of an extremity;
a plurality of thermal imagers disposed within the enclosure comprising,
a first thermal imager disposed at the top side is configured to detect a nail surface temperature,
a second thermal imager disposed at the bottom side is configured to detect a pad temperature, and
a third thermal imager disposed at the first side and
a fourth thermal imager disposed at the second side
the plurality configured to detect at least one of a nail surface or finger pad temperature; and
a computing device in communication with the plurality of thermal imagers and the extremity detection unit is configured to analyze nail surface temperatures and pad temperatures from the plurality of thermal imagers to detect the presence of a pathological condition of the user.

15. The disease detection device of claim 14, wherein the enclosure comprises non-reflective, high-emissivity, uniform inner surface.

16. The disease detection device of claim 14, further comprises means to measure an ambient temperature by imaging the inner surface of the enclosure.

17. The disease detection device of claim 14, wherein the plurality of thermal imagers are configured to be re-calibrated to a single standard temperature by imaging the inner surface of the enclosure.

18. The disease detection device of claim 14, wherein the detection unit is at least any one of a light beam interruption device, ultrasonic digit detection unit, and capacitive sensing unit.

19. A method to detect a presence of pathological condition of user, comprising steps of:
providing a disease detection device, comprising
a measurement area for receiving an extremity of a user,
a detection unit for sensing the presence of an extremity in the measurement area,
a plurality of thermal imagers disposed within the housing comprising,
a first thermal imager configured to detect a nail surface temperature,
a second thermal imager configured to detect a pad temperature, and
a computing device in communication with the detection unit and at least two thermal imagers configured to provide instruction to user to aid during diagnosis;
providing instruction to the user to undergo a vasoconstricted state;
placing a first unclasped extremity within the measurement area in unclasped state for radiometrically imaging via the at least two thermal imagers;
placing a second unclasped extremity within the measurement area for radiometrically imaging via the at least two thermal imagers;
transmitting a thermal image data of the first and second extremity of the user to the computing device;
comparing the thermal image data of the first and second extremity with a nominal body core temperature of a user by the computing device; and
providing result regarding the presence of pathological condition to the user.

20. The method according to claim 19, wherein the user undergoes the vasoconstricted state by resting in an area of ambient temperature at least 20° F. (11.1° ° C.) below the nominal body temperature of the user.
